(19) United States
(12) Patent Application Publication
(10) Pub. No.: US 2024/0000557 A1
(43) Pub. Date: Jan. 4, 2024

(54) ELECTRIC TOOTHBRUSH AND COMBINATION STRUCTURE THEREOF

(71) Applicant: Proxihealthcare Inc., Ulsan (KR)

(72) Inventor: Young Wook Kim, Seoul (KR)

(21) Appl. No.: 18/240,386

(22) Filed: Aug. 31, 2023

Related U.S. Application Data
(63) Continuation of application No. PCT/KR22/19739, filed on Dec. 6, 2022.

(30) Foreign Application Priority Data
Apr. 18, 2022 (KR) 10-2022-0047773

(51) Int. Cl. A61C 17/22; A61C 17/34

(52) U.S. Cl. CPC A61C 17/222; A61C 17/34; A61C 2204/002

(57) ABSTRACT

An electric toothbrush includes a head unit and a handle unit detachably combined with the head unit and supplying a driving voltage to the head unit in response to a user's manipulation. The handle unit includes an insertion member including a base portion and one or more protrusion portions protruding from the base portion, and a plurality of connection terminals having a portion positioned to protrude outward from the insertion member, and electrically connected to the head unit when the head unit and the handle unit are combined. The head unit includes a receiving member including an inlet part recessed to a depth into which the insertion member is inserted, a separation preventing part protruding from an inner wall surface of the inlet part and located to overlap with the protrusion portion when the insertion member is inserted into the inlet part and then rotated at a certain angle, and a plurality of insertion parts formed to pass through a bottom surface of the inlet part; and an electrode assembly installed adjacent to the receiving member inside the head unit, and when the handle unit and the head unit are combined, passing through the plurality of insertion parts and electrically connected to ends of the plurality of connection terminals located inside the head unit.

### CROSS REFERENCE TO RELATED APPLICATION

This application is a Bypass Continuation of International Application No. PCT/KR2022/019739, filed on Dec. 6, 2022, and claims priority from and the benefit of Korean Patent Application No. 10-2022-0047773, filed on Apr. 18, 2022, each of which is incorporated by reference for all purposes as if fully set forth herein.

### BACKGROUND

### Field

Embodiments of the invention relate generally to an electric toothbrush, and more specifically, to an electric toothbrush capable of effectively removing dental plaque.

### Discussion of the Background

Dental plaque is a sticky and transparent film that adheres to the surface of teeth. The dental plaque is formed as numerous germs (bacteria) living in the mouth adhere to certain components in saliva. The dental plaque may be formed not only on and around the teeth, but also around prostheses, braces, and dentures.

When the dental plaque in the form of a very thin and transparent film is created, the bacteria in the plaque proliferate and also increase exponentially using the sugar supplied when food is consumed. The acidic substances produced by the bacteria in the plaque dissolve the lime in the teeth, causing tooth decay, and the toxins cause inflammation in the gums.

The dental plaque itself is difficult to see with the naked eye, and it mainly occurs in deep valleys of teeth, narrow gaps between teeth, and narrow gaps between teeth and gums. Because the plaque causes problems to teeth and surrounding tissues in such a small space, it is important to remove the plaque without missing every corner.

The above information disclosed in this Background section is only for understanding of the background of the inventive concepts, and, therefore, it may contain information that does not constitute prior art.

### SUMMARY

Applicant recognized that there is a problem in that it is difficult to effectively remove such plaque using only a conventional toothbrush.

Electric toothbrush constructed according to illustrative implementations of the invention is capable of preventing tooth decay and periodontal disease through the removal of dental plaque.

Further, electric toothbrush constructed according to illustrative implementations of the invention can be used by replacing a head unit where bristles are located.

Additional features of the inventive concepts will be set forth in the description which follows, and in part will be apparent from the description, or may be learned by practice of the inventive concepts.

According to one aspect of the invention, an electric toothbrush includes a head unit; and a handle unit detachably combined with the head unit and supplying a driving voltage to the head unit in response to a user's manipulation, wherein the handle unit includes: an insertion member, which is inserted into the head unit when the head unit and the handle unit are combined, including a base portion and one or more protrusion portions protruding from the circumferential surface of the base portion; and a plurality of connection terminals having a portion positioned to protrude outward from the insertion member, and electrically connected to the head unit, and wherein the head unit includes: a receiving member including an inlet part recessed to a depth into which the insertion member is inserted, a separation preventing part protruding from an inner wall surface of the inlet part and located to overlap with the protrusion portion when the insertion member is inserted into the inlet part and then rotated at a certain angle, and a plurality of insertion parts, which is formed to pass through a bottom surface of the inlet part and allows the plurality of connection terminals to be mechanically engaged with the head unit when the handle unit and the head unit rotate relative to each other in a state where the plurality of connection terminals are inserted; and an electrode assembly installed adjacent to the receiving member inside the head unit, and when the handle unit and the head unit are combined, passing through the plurality of insertion parts and electrically connected to ends of the plurality of connection terminals located inside the head unit.

In addition, the handle unit may include a handle housing combined with the insertion member; a battery installed in the handle housing; a main circuit board on which a signal supply unit for generating a driving voltage using a voltage of the battery is mounted; and a connection module including a contact block made of a non-conductive material, and a contact terminal made of a conductive material and installed to surround the contact block, one side of the contact terminal being in contact with the main circuit board and the other side being in contact with the connection terminal so that the main circuit board and the plurality of connection terminals are electrically connected.

In addition, the head unit may include a head housing including a head portion having one opened side and a combining portion communicating with the head portion; an auxiliary circuit board installed inside the head housing; a head cover closing an opened area of the head portion; bristles combined with the head cover; and first and second electrodes positioned to correspond to the head portion on the auxiliary circuit board to generate an electric field, wherein the electrode assembly is positioned adjacent to a part of the combining portion combined with the handle unit, and is combined with the auxiliary circuit board.

In addition, the electrode assembly may include an insulating member made of a non-conductive material and having a through-hole into which a portion of the auxiliary circuit board is inserted; and a conductive member made of a conductive material, having one side installed near the through-hole of the insulating member and electrically connected to the auxiliary circuit board, and having the other side positioned adjacent to the receiving member and electrically connected to the plurality of connection terminals when the handle unit and the head unit are combined with each other.

In addition, the conductive member may include a gripping part gripping a portion of the insulating member where the through-hole is formed; a pressing part formed by bending a portion of the gripping part that contacts the auxiliary circuit board, and pressing the auxiliary circuit board; and a contact part extending from the gripping part and coming into contact with the connection terminal.

In addition, an end portion of the contact part may be spaced apart from the insulating member, and a middle portion of the contact part may be formed to protrude toward the plurality of connection terminals.

In addition, at least one bar-shaped extension may be formed in the auxiliary circuit board to be inserted into a through-hole of the insulating member.

In addition, the first electrode and the second electrode may be located to be spaced apart from each other along a length direction of the auxiliary circuit board.

In addition, the head unit may further include a light emitting element that is installed on the auxiliary circuit board and emits light to outside.

In addition, the head cover may be made of a transparent material.

In addition, the handle unit may further include a sealing member positioned at a portion of the handle housing that is in contact with the head unit, the sealing member sealing between the handle housing and the head unit.

In addition, the plurality of connection terminals may include a first connection terminal; and one or more second connection terminals spaced apart from the first connection terminal.

In addition, the plurality of insertion parts may include a first insertion part formed to pass through a bottom surface of the inlet part, and allowing the first connection terminal to be electrically connected to the electrode assembly; and at least one second insertion part formed to pass through the bottom surface of the inlet part, formed in an arc shape so that the second connection terminal inserted therein moves from one end to the other end when the handle unit and the head unit rotate relative to each other, and allowing the second connection terminal to be electrically connected to the electrode assembly.

In addition, the second insertion part may include a first region where the one or more second connection terminals are positioned when the insertion member is inserted into the inlet part of the receiving member; a second region positioned to face a middle portion of the contact part formed to protrude toward the plurality of connection terminals, and providing a region where the at least one second connection terminal moved from the first region is finally positioned when the insertion member is inserted into the inlet part of the receiving member and then rotated at a certain angle; and a position fixing protrusion protruding from a portion of the second insertion part where the second region is located, wherein when an external force equal to or greater than a threshold is applied in a state where the second connection terminal is located in the second region, the position fixing protrusion enables the second connection terminal to be separated from the second region, and wherein when an external force equal to or greater than a threshold is not applied, the second connection terminal is continuously positioned in the second region.

According to another aspect of the invention, a combination structure of an electric toothbrush includes a head unit and a handle unit detachably combined with each other, wherein: the handle unit including: an insertion member including a plurality of protrusion portions; and a first connection terminal and a pair of second connection terminals passing through the insertion member and exposed to outside; and the head unit including: a receiving member positioned at a combining portion with the handle unit, the receiving member including: a plurality of separation preventing parts; a first insertion part located in a center and into which the first connection terminal is inserted; and a pair of second insertion parts positioned symmetrically with respect to the first insertion part and having a first region and a second region where the pair of second connection terminals are to be located, wherein in an initial state where the insertion member is inserted into the receiving member, the second connection terminal is located in the first region, and when the handle unit rotates with respect to the head unit, the second connection terminal located in the first region moves to the second region, and the protrusion portions are respectively inserted into the separation preventing parts

According to the present disclosure, it is possible to provide an electric toothbrush that allows, when replacement of a head unit is required, the head unit to be separated from a handle unit and easily replaced with a new head unit.

In addition, according to the present disclosure, it is possible to provide an electric toothbrush capable of effectively removing dental plaque.

In addition, according to the present disclosure, it is possible to provide an electric toothbrush capable of preventing tooth decay and periodontal disease through the removal of dental plaque.

In addition, according to the present disclosure, it is possible to provide an electric toothbrush capable of stably maintaining a combining state between the handle unit and the head unit.

In addition, according to the present disclosure, it is possible to provide an electric toothbrush capable of preventing moisture from entering from the outside.

It is to be understood that both the foregoing general description and the following detailed description are illustrative and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate illustrative embodiments of the invention, and together with the description serve to explain the inventive concepts.

FIG. 1 is a perspective view showing an electric toothbrush according to an embodiment of the present disclosure;

FIG. 2 is a perspective view showing a state in which a head unit and a handle unit included in an electric toothbrush according to an embodiment of the present disclosure are being combined;

FIG. 3 is a perspective view showing a separated state of the electric toothbrush shown in FIG. 1;

FIG. 4 is a view of the handle unit of FIG. 3 viewed from above;

FIG. 5 is a cross-sectional view taken along line A-A in the handle unit of FIG. 3;

FIG. 6 is an exploded perspective view showing the insertion member, the main circuit board, the connection terminal, and the connection module extracted from the handle unit of FIG. 3;

FIG. 7 is a view showing the head unit extracted from FIG. 3;

FIG. 8 is a cross-sectional view taken along line B-B in the head unit of FIG. 5;

FIG. 9 is an exploded perspective view showing the head unit of FIG. 5;

FIG. 10 is a view of the head unit of FIG. 5 viewed from the Z direction;

FIG. 11 is a view showing a process of combining an insertion member and a receiving member;

FIG. 12 is a view showing an electrode assembly extracted from the head unit of FIG. 9;

FIG. 13 is a cross-sectional view taken along line C-C in the electrode assembly of FIG. 12;

FIG. 14 is a front view showing an electric toothbrush according to an embodiment of the present disclosure;

FIG. 15 is a view showing a signal supply unit mounted on a main circuit board;

FIGS. 16 to 18 are views showing waveforms of signals according to embodiments of the present disclosure;

FIGS. 19 and 20 are views showing a biofilm removal effect of a driving signal generated by mixing an AC signal and a DC signal;

FIGS. 21 and 22 are views showing a modified example of a signal supply unit; and

FIG. 23 is a perspective view showing a use example of an electric toothbrush according to an embodiment of the present disclosure.

### DETAILED DESCRIPTION

In the following description, for the purposes of explanation, numerous specific details are set forth in order to provide a thorough understanding of various embodiments or implementations of the invention. As used herein “embodiments” and “implementations” are interchangeable words that are non-limiting examples of devices or methods employing one or more of the inventive concepts disclosed herein. It is apparent, however, that various embodiments may be practiced without these specific details or with one or more equivalent arrangements. In other instances, well-known structures and devices are shown in block diagram form in order to avoid unnecessarily obscuring various embodiments. Further, various embodiments may be different, but do not have to be exclusive. For example, specific shapes, configurations, and characteristics of an embodiment may be used or implemented in another embodiment without departing from the inventive concepts.

Unless otherwise specified, the illustrated embodiments are to be understood as providing illustrative features of varying detail of some ways in which the inventive concepts may be implemented in practice. Therefore, unless otherwise specified, the features, components, modules, layers, films, panels, regions, and/or aspects, etc. (hereinafter individually or collectively referred to as “elements”), of the various embodiments may be otherwise combined, separated, interchanged, and/or rearranged without departing from the inventive concepts.

The use of cross-hatching and/or shading in the accompanying drawings is generally provided to clarify boundaries between adjacent elements. As such, neither the presence nor the absence of cross-hatching or shading conveys or indicates any preference or requirement for particular materials, material properties, dimensions, proportions, commonalities between illustrated elements, and/or any other characteristic, attribute, property, etc., of the elements, unless specified. Further, in the accompanying drawings, the size and relative sizes of elements may be exaggerated for clarity and/or descriptive purposes. When an embodiment may be implemented differently, a specific process order may be performed differently from the described order. For example, two consecutively described processes may be performed substantially at the same time or performed in an order opposite to the described order. Also, like reference numerals denote like elements.

When an element, such as a layer, is referred to as being “on,” “connected to,” or “coupled to” another element or layer, it may be directly on, connected to, or coupled to the other element or layer or intervening elements or layers may be present. When, however, an element or layer is referred to as being “directly on,” “directly connected to,” or “directly coupled to” another element or layer, there are no intervening elements or layers present. To this end, the term “connected” may refer to physical, electrical, and/or fluid connection, with or without intervening elements. Further, the D1-axis, the D2-axis, and the D3-axis are not limited to three axes of a rectangular coordinate system, such as the x, y, and z-axes, and may be interpreted in a broader sense. For example, the D1-axis, the D2-axis, and the D3-axis may be perpendicular to one another, or may represent different directions that are not perpendicular to one another. For the purposes of this disclosure, “at least one of X, Y, and Z” and “at least one selected from the group consisting of X, Y, and Z” may be construed as X only, Y only, Z only, or any combination of two or more of X, Y, and Z, such as, for instance, XYZ, XYY, YZ, and ZZ. As used herein, the term “and/or” includes any and all combinations of one or more of the associated listed items.

Although the terms “first,” “second,” etc. may be used herein to describe various types of elements, these elements should not be limited by these terms. These terms are used to distinguish one element from another element. Thus, a first element discussed below could be termed a second element without departing from the teachings of the disclosure.

Spatially relative terms, such as “beneath,” “below,” “under,” “lower,” “above,” “upper,” “over,” “higher,” “side” (e.g., as in “sidewall”), and the like, may be used herein for descriptive purposes, and, thereby, to describe one elements relationship to another element(s) as illustrated in the drawings. Spatially relative terms are intended to encompass different orientations of an apparatus in use, operation, and/or manufacture in addition to the orientation depicted in the drawings. For example, if the apparatus in the drawings is turned over, elements described as “below” or “beneath” other elements or features would then be oriented “above” the other elements or features. Thus, the term “below” can encompass both an orientation of above and below. Furthermore, the apparatus may be otherwise oriented (e.g., rotated 90 degrees or at other orientations), and, as such, the spatially relative descriptors used herein interpreted accordingly.

The terminology used herein is for the purpose of describing particular embodiments and is not intended to be limiting. As used herein, the singular forms, “a,” “an,” and “the” are intended to include the plural forms as well, unless the context clearly indicates otherwise. Moreover, the terms “comprises,” “comprising,” “includes,” and/or “including,” when used in this specification, specify the presence of stated features, integers, steps, operations, elements, components, and/or groups thereof, but do not preclude the presence or addition of one or more other features, integers, steps, operations, elements, components, and/or groups thereof. It is also noted that, as used herein, the terms “substantially,” “about,” and other similar terms, are used as terms of approximation and not as terms of degree, and, as such, are utilized to account for inherent deviations in measured, calculated, and/or provided values that would be recognized by one of ordinary skill in the art.

Various embodiments are described herein with reference to sectional and/or exploded illustrations that are schematic illustrations of idealized embodiments and/or intermediate structures. As such, variations from the shapes of the illustrations as a result, for example, of manufacturing techniques and/or tolerances, are to be expected. Thus, embodiments disclosed herein should not necessarily be construed as limited to the particular illustrated shapes of regions, but are to include deviations in shapes that result from, for instance, manufacturing. In this manner, regions illustrated in the drawings may be schematic in nature and the shapes of these regions may not reflect actual shapes of regions of a device and, as such, are not necessarily intended to be limiting.

Unless otherwise defined, all terms (including technical and scientific terms) used herein have the same meaning as commonly understood by one of ordinary skill in the art to which this disclosure is a part. Terms, such as those defined in commonly used dictionaries, should be interpreted as having a meaning that is consistent with their meaning in the context of the relevant art and should not be interpreted in an idealized or overly formal sense, unless expressly so defined herein.

Hereinafter, embodiments of the present disclosure will be described in detail with reference to the accompanying drawings. However, the present disclosure is not limited to the embodiments disclosed below and may be implemented in various different forms. Also, it should be understood that all modifications, equivalents, or replacements thereof are included within the subject matter and scope of the present disclosure.

Now, an electric toothbrush according to an embodiment of the present disclosure will be described with reference to drawings related to the embodiment.

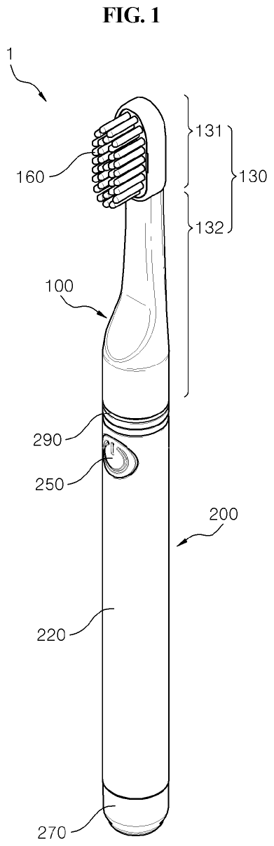

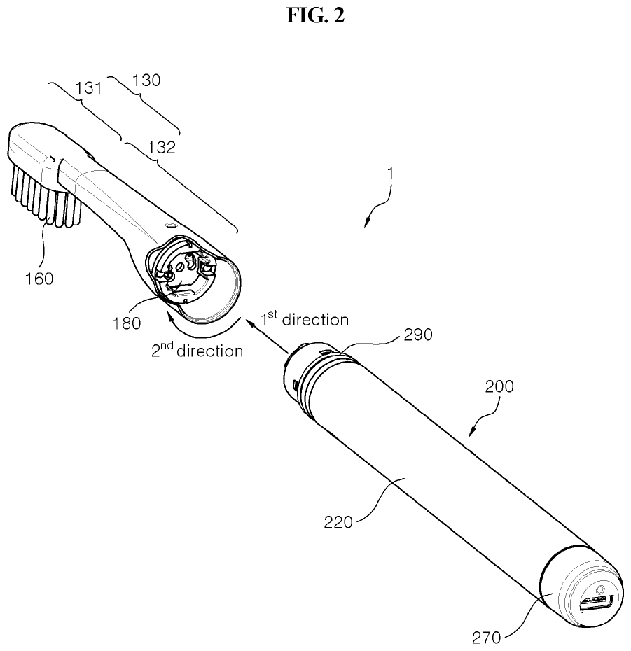

FIG. 1 is a perspective view showing an electric toothbrush according to an embodiment of the present disclosure, and FIG. 2 is a perspective view showing a state in which a head unit and a handle unit included in an electric toothbrush according to an embodiment of the present disclosure are being combined.

Referring to FIGS. 1 and 2, an electric toothbrush 1 according to an embodiment of the present disclosure may include a head unit 100 and a handle unit 200.

At least a portion of the head unit 100 may be inserted into the user's oral cavity to remove dental plaque. The head unit 100 may be detachably combined with the handle unit 200 to be described later. Therefore, when replacement of the head unit 100 is required due to deterioration or the like, the user can easily replace the existing head unit 100 with a new head unit 100. A detailed description of the head unit 100 for this purpose will be described later.

The handle unit 200 is a main body of the electric toothbrush 1 and may be designed in a form of allowing the user to hold in use. The handle unit 200 is detachably combined with the head unit 100 and can supply a driving voltage to the head unit 100 in response to a user's manipulation. In addition, when the handle unit 200 is partially inserted into the head unit 100 and then rotated with respect to the head unit 100, it may be electrically connected to and mechanically engaged with the head unit 100.

Specifically, as shown in FIG. 2, in a state where the handle unit 200 and the head unit 100 are positioned in a line, the user moves the handle unit 200 in a first direction to insert the tip of the handle unit 200 into the head unit 100 and then rotates the handle unit 200 in a second direction, so that the head unit 100 and the handle unit 200 can be combined with each other. A process of separating the handle unit 200 and the head unit 100 can be performed in the reverse order of the above mentioned assembling process.

Unlike the above, in an electric toothbrush in which the handle unit 200 and the head unit 100 are integrally formed, when bristles 160 of the head unit 100 are damaged due to repeated use, the entirety of the toothbrush should be replaced. However, in the electric toothbrush 1 according to the embodiment of the present disclosure, when the bristles 160 of the head unit 100 are damaged due to repetitive use, it is possible to separate the existing head unit 100 from the handle unit 200 and then use a new head unit 100 by combining it with the handle unit 200.

Hereinafter, the handle unit 200 and the head unit 100 included in the electric toothbrush 1 according to the embodiment of the present disclosure will be described in detail with reference to the drawings.

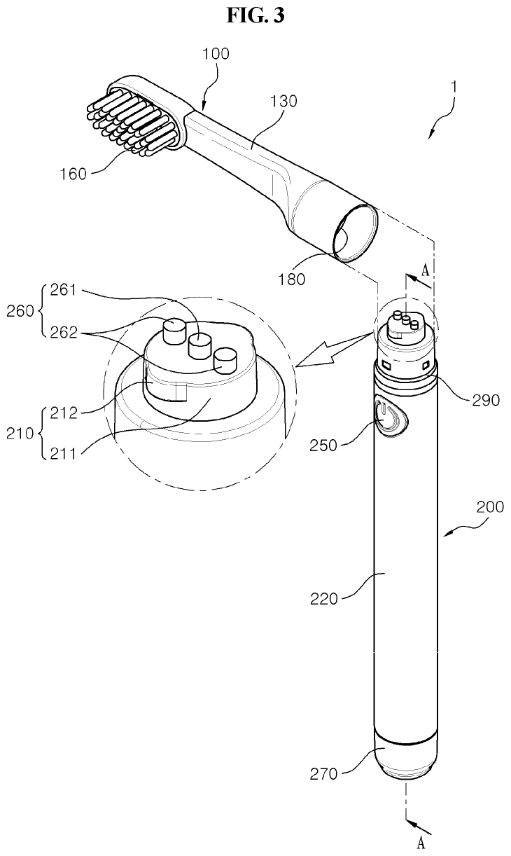

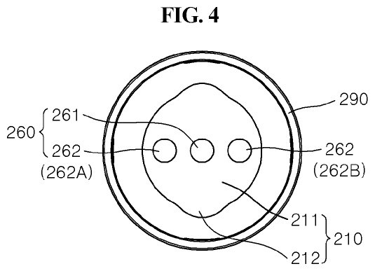

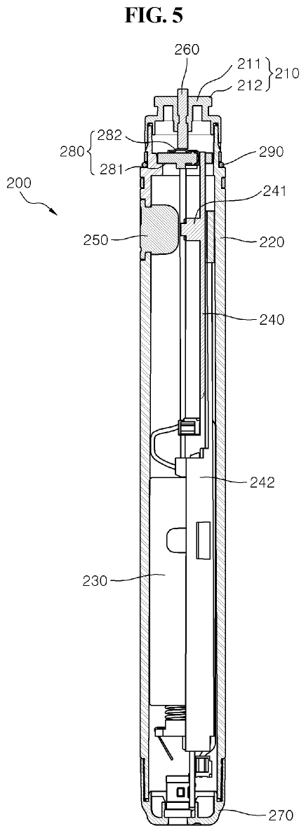

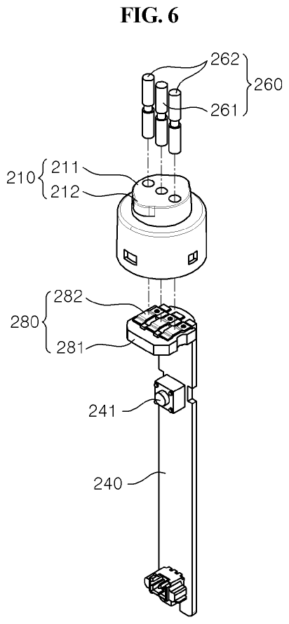

FIG. 3 is a perspective view showing a separated state of the electric toothbrush shown in FIG. 1, FIG. 4 is a view of the handle unit of FIG. 3 viewed from above, FIG. 5 is a cross-sectional view taken along line A-A in the handle unit of FIG. 3, and FIG. 6 is an exploded perspective view showing the insertion member, the main circuit board, the connection terminal, and the connection module extracted from the handle unit of FIG. 3.

Referring to FIGS. 3 to 6, the above-described handle unit 200 may include, for example, an insertion member 210, a handle housing 220, a battery 230, a main circuit board 240, a manipulation member 250, a connection terminal 260, and a connection module 280.

The insertion member 210 may be inserted into the head unit 100 when the handle unit 200 and the head unit 100 are combined. To this end, the insertion member 210 may include, for example, a base portion 211 and a protrusion portion 212.

The base portion 211 may be formed in a pillar shape. The shape of the base portion 211 may be, for example, a cylindrical shape. The connection terminal 260 to be described later may be installed to protrude outwardly through the base portion 211.

The protrusion portion 212 includes one or more protrusion portions 212 formed in a plate shape and protruding from the circumferential surface of the base portion 211. The protrusion portion 212 may protrude from the circumferential surface of an upper side of the base portion 211. When the handle unit 200 and the head unit 100 are combined, the protrusion portion 212 may be engaged with a portion of the head unit 100 so that the head unit 100 can be combined with the handle unit 200. This will be described in detail when the head unit 100 is described.

The handle housing 220 may be combined with the insertion member 210. The handle housing 220 can accommodate the battery 230, the main circuit board 240, and the connection terminal 260 and protect them from external contact.

The battery 230 may be installed in the handle housing 220 and can supply power to the main circuit board 240 to be described later. A first sealing member 270 for replacement of the battery 230 may be separately installed in the handle housing 220, but this is not a limitation.

The battery 230 may be, for example, a primary battery or a secondary battery. When the battery 230 is a primary battery, the user may periodically replace the battery 230, and when the battery 230 is a secondary battery, charging may be performed through various charging schemes.

For example, the battery 230 may be charged through a wireless charging scheme or a wired charging scheme, and it may be separated from the handle housing 220 and then charged through a separate charging device.

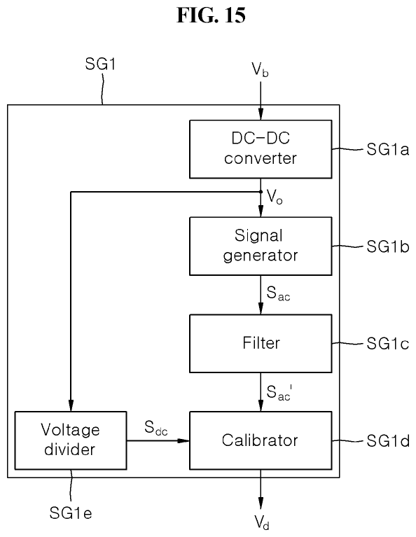

The main circuit board 240 may provide a driving voltage. To this end, a signal supply unit (SG1, see FIG. 15) that generates the driving voltage using the voltage of the battery 230 may be mounted on the main circuit board 240. A detailed description of the main circuit board 240 will be described later.

The manipulation member 250 may control the supply of power from the battery 230 to the main circuit board 240. The manipulation member 250 may be installed on a portion of the handle housing 220.

The manipulation member 250 may be, for example, a push button or a touch sensor. When the manipulation member 250 is a push button, a switch 241 that detects the push of the manipulation member 250 may be mounted on the main circuit board 240. When the user presses the manipulation member 250, the switch 241 detects the push of the manipulation member 250 to allow the electric toothbrush 1 to be turned on/off.

The user can turn on the power of the electric toothbrush 1 by pressing the manipulation member 250 when brushing teeth. Then, the driving voltage generated from the handle unit 200 can be applied to a first electrode 110 and/or a second electrode 120 of the head unit 100 to generate an electric field for removing dental plaque.

The connection terminal 260 is electrically connected to the main circuit board 240. A portion of the connection terminal 260 may be positioned to protrude outward from the insertion member 210. That is, based on a direction shown in FIG. 5, the connection terminal 260 may be electrically connected at a lower end thereof to the main circuit board 240 and installed to pass through the insertion member 210.

Here, the connection terminal 260 may be directly connected to the main circuit board 240 or may be indirectly connected to the main circuit board 240 through a connection module 280 to be described later. A detailed description of the connection module 280 will be described later. However, a connection method between the connection terminal 260 and the main circuit board 240 is not limited to a specific method because various methods may be applied.

When the head unit 100 and the handle unit 200 are combined, the connection terminal 260 can be electrically connected to the head unit 100 to deliver the driving voltage of the main circuit board 240 to the head unit 100.

The connection terminal 260 may be one or more. When there are a plurality of connection terminals 260, the plurality of connection terminals 260 may include, for example, a first connection terminal 261 and one or more second connection terminals 262 positioned spaced apart from the first connection terminal 261.

Here, the second connection terminal 262 may be two. In this case, two second connection terminals 262A and 262B may be positioned symmetrically with respect to the first connection terminal 261. Because the number of connection terminals 260 may be changed depending on the design of the electric toothbrush 1, it is not limited to a specific number.

Meanwhile, the connection terminal 260 may be formed of a conductive material such as metal and may have a rod or stick shape having a predetermined length. In addition, the insertion member 210 may be made of an insulating material such as, for example, non-conductive plastic to prevent the first connection terminal 261 and the plurality of second connection terminals 262 from being energized with each other.

The connection module 280 electrically connects the main circuit board 240 and the plurality of connection terminals 260. For this, the connection module 280 may include a contact block 281 and one or more contact terminals 282.

The contact block 281 is made of a non-conductive material. A portion of the contact block 281 may be combined with the main circuit board 240, and another portion may be positioned adjacent to ends of the plurality of connection terminals 260. The contact block 281 may have the same shape as, for example, the insulating member 171 included in the electrode assembly 170 to be described later, but this is not a limitation.

The contact terminal 282 is made of a conductive material and installed to surround the contact block 281. One side of the contact terminal 282 is in contact with the main circuit board 240, and the other side is in contact with the connection terminal 260.

The contact terminal 282 may be made of an elastically deformable material such as a leaf spring. Therefore, combining force with the contact block 281 can be improved, and contact stability upon contact with the connection terminal 260 can be improved.

Meanwhile, the number of contact terminals 282 may correspond to the number of connection terminals 260. For example, when the number of connection terminals 260 is three, the number of contact terminals 282 may also be three, and the connection terminals 260 and the contact terminal 282 may be in contact with each other in a one-to-one correspondence. The contact terminal 282 may have the same shape as, for example, the conductive member 172 included in the electrode assembly 170 to be described later, but this is not a limitation.

The main circuit board 240 and the connection terminal 260 can always maintain electrically stable connection through the connection module 280 as described above.

Meanwhile, the handle unit 200 may further include a second sealing member 290.

The second sealing member 290 may be positioned at a portion of the handle housing 220 that is in contact with the head unit 100, and can seal between the handle housing 220 and the head unit 100. The second sealing member 290 may have a ring shape and may be formed of a material such as rubber or silicon. The second sealing member 290 may be fitted into a portion of the handle housing 220. For example, the second sealing member 290 may be positioned adjacent to the insertion member 210 in the handle housing 220.

While the user uses the electric toothbrush 1, saliva or toothpaste may flow from the head unit. The second sealing member 290 blocks a material such as saliva or toothpaste from entering between the handle housing 220 and the head unit 100, thereby preventing the head unit 100 or the handle unit 200 from being damaged by moisture.

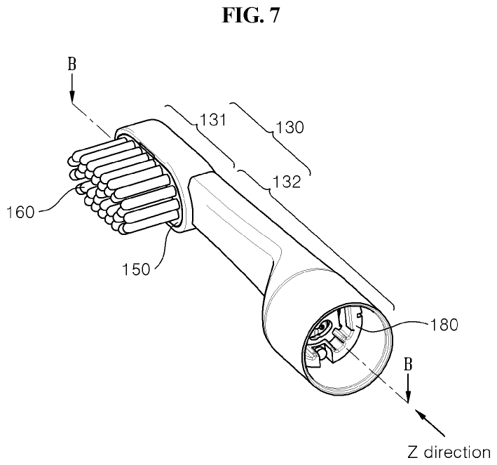

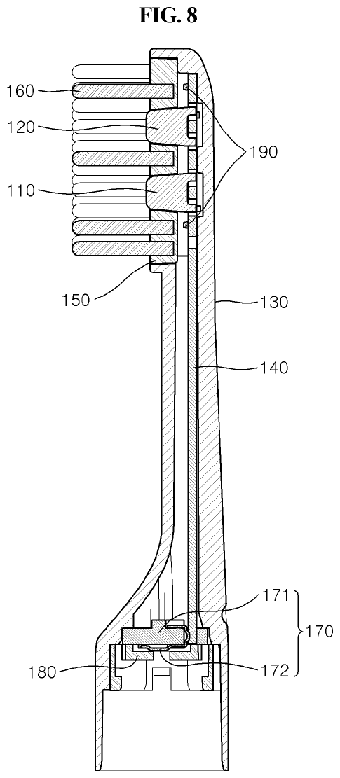

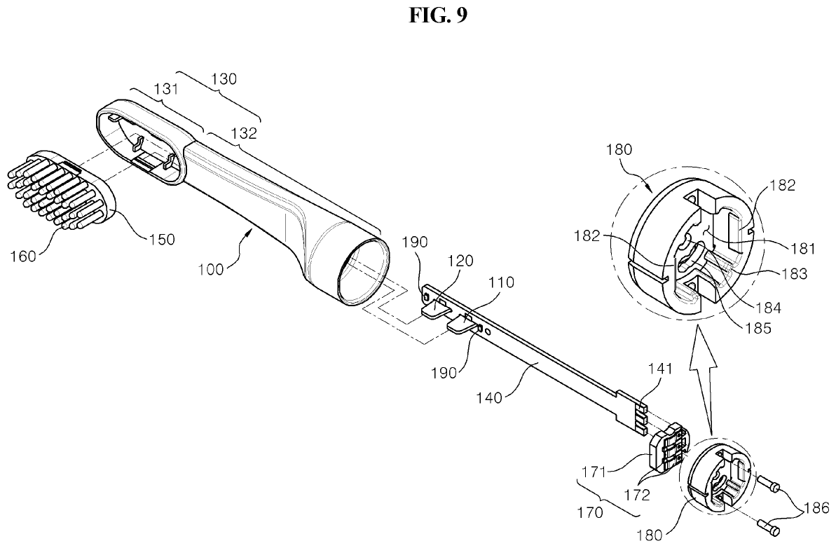

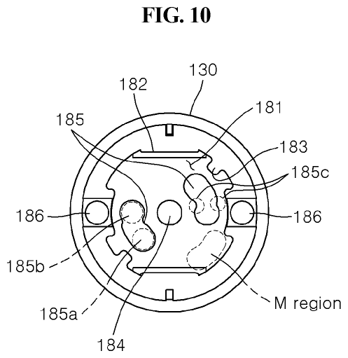

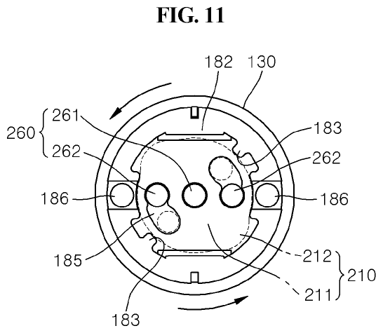

FIG. 7 is a view showing the head unit extracted from FIG. 3, FIG. 8 is a cross-sectional view taken along line B-B in the head unit of FIG. 5, FIG. 9 is an exploded perspective view showing the head unit of FIG. 5, FIG. 10 is a view of the head unit of FIG. 5 viewed from the Z direction, and FIG. 11 is a view showing a process of combining an insertion member and a receiving member.

Referring to FIGS. 7 to 11, the above-described head unit 100 may include, for example, a head housing 130, an auxiliary circuit board 140, first and second electrodes 110 and 120, a head cover 150, bristles 160, an electrode assembly 170, and a receiving member 180.

The head housing 130 may include a head portion 131 having one opened side and a combining portion 132 communicating with the head portion 131. The head portion 131 and the combining portion 132 may be integrally formed.

The combining portion 132 may be combined with the handle unit 200. In the head portion 131, the head cover 150, the bristles 160, and the first and second electrodes 110 and 120, which are to be described later, may be located.

The auxiliary circuit board 140 may be installed inside the head housing 130. At least one bar-shaped extension 141 may be formed in the auxiliary circuit board 140 to be inserted into a through-hole 171a of an insulating member 171.

The extensions 141 may be provided to correspond to the number of connection terminals 260. For example, when the above-described connection terminal 260 includes one first connection terminal 261 and two second connection terminals 262, the number of extensions 141 may be three. A conductive line (not shown) may be formed in each extension 141, and each conductive line may be electrically connected to the connection terminal 260 through the electrode assembly 170 to be described later.

The head cover 150 may close an opened area of the head portion 131. To this end, the head cover 150 may be formed in a shape corresponding to an opening of the head portion 131.

The bristles 160 may be combined with the head cover 150. The bristles 160 may be inserted into and fixed to a plurality of bristle holes (not shown) formed on the outer surface of the head cover 150. The arrangement structure, number, size, etc. of these bristles 160 are not particularly limited and may be changed in various forms.

The first electrode 110 and the second electrode 120 may be positioned to correspond to the head portion 131 on the auxiliary circuit board 140 to generate an electric field. The first electrode 110 and the second electrode 120 may be mounted on the auxiliary circuit board 140.

The first electrode 110 and the second electrode 120 may generate an electric field by receiving electrical energy from the handle unit 200. Because this electric field can weaken the dental plaque structure, the user can effectively remove dental plaque in the oral cavity by using the electric toothbrush 1 according to an embodiment of the present disclosure.

The first electrode 110 and the second electrode 120 may be formed of a material such as, but is not limited to, brass, aluminum, conducting polymer, conducting silicon, or stainless steel. Any material having conductivity may also be used as the material of the electrode.

The first electrode 110 and the second electrode 120 may be set as a positive electrode and a negative electrode, respectively. The first electrode 110 and the second electrode 120 may be formed at a height sufficient to protrude from the head cover 150 to the outside. The heights of the first and second electrodes 110 and 120 may be changed depending on the design of the electric toothbrush 1 and are not limited to specific values.

The electrode assembly 170 is installed adjacent to the receiving member 180 inside the head unit 100, and when the handle unit 200 and the head unit 100 are combined, it may pass through the plurality of insertion parts 184 and 185 and be electrically connected to the ends of the plurality of connection terminals 260 located inside the head unit 100. For this, the electrode assembly 170 may be positioned adjacent to a part of the combining portion 132 combined with the handle unit 200, and combined with the auxiliary circuit board 140.

That is, the above-described auxiliary circuit board 140 may receive the driving voltage of the handle unit 200 from the electrode assembly 170 and transfer it to the above-described first and second electrodes 110 and 120. A detailed description of the electrode assembly 170 will be described later.

The receiving member 180 may be positioned adjacent to the electrode assembly 170 and combined with an end of the combining portion 132 where the handle unit 200 is combined. The receiving member 180 may be fastened to the electrode assembly 170 or the head housing 130 by a separate bolt 186. When the handle unit 200 and the head unit 100 are combined, the receiving member 180 may be mechanically fastened to the handle unit 200.

In addition, the receiving member 180 may allow a portion of the electrode assembly 170 to be exposed to the outside. Accordingly, the above-described connection terminal 260 may pass through the receiving member 180 and come into contact with the electrode assembly 170, and a detailed description thereof will be described later.

Meanwhile, the receiving member 180 as described above may include, for example, an inlet part 181, a separation preventing part 182, and a plurality of insertion parts 184 and 185.

The inlet part 181 may have a shape recessed to a depth into which the insertion member 210 can be inserted.

The separation preventing part 182 may be formed in a plate shape and protrude from the inner wall surface of the inlet part 181. When the insertion member 210 is inserted into the inlet part 181 and then rotated at a certain angle, the separation preventing part 182 may be placed outside the protrusion portion 212 to overlap with the protrusion portion 212.

The plurality of insertion parts 184 and 185 may be formed to pass through the bottom surface of the inlet part 181. When the handle unit 200 and the head unit 100 rotate relative to each other in a state where the plurality of connection terminals 260 are inserted, the plurality of insertion parts 184 and 185 may be mechanically engaged with the plurality of connection terminals 260. By this engagement between the plurality of insertion parts 184 and 185 and the plurality of connection terminals 260, the head unit 100 and the handle unit 200 may be mechanically combined with each other.

Meanwhile, the receiving member 180 may further include a support part 183.

The support part 183 protrudes from the inner wall surface of the inlet part 181. The support part 183 may improve the fixing force with respect to the base portion 211 by supporting the circumferential surface of the base portion 211. In addition, when the handle unit 200 moves toward the head unit 100 in the first direction (see FIG. 2), the support part 183 may allow the protrusion portion 212 of the insertion member 210 to be inserted into an M region of the inlet part 181 while preventing insertion into other regions.

Meanwhile, the head unit 100 may further include a light emitting element 190.

The light emitting element 190 may be installed on the auxiliary circuit board 140 and can emit light to the outside.

The head cover 150 included in the aforementioned head unit 100 may be made of a transparent material. The material of the head cover 150 may be, for example, polycarbonate, or may be an acrylic plate for diffusing light. The head cover 150 may have fine protrusions formed on the outer surface thereof to facilitate light diffusion. Also, the head housing 130 may be made of a transparent material so that the head unit 100 can emit light when the electric toothbrush 1 operates.

Meanwhile, one of the three connection terminals 260 may be connected to a ground terminal of the main circuit board 240, another may receive a driving voltage for generating an electric field from the main circuit board 240, and the other may receive power for driving the light emitting element 190 from the main circuit board 240. For example, one of the two second connection terminals 262 may be connected to the ground terminal of the main circuit board 240, and the other may receive the driving voltage from the main circuit board 240. In addition, the first connection terminal 261 may receive power from the main circuit board 240 and transmit it to the light emitting element 190.

Meanwhile, as described above, the receiving member 180 allows the connection terminal 260 included in the aforementioned handle unit 200 to pass through and be electrically connected to the head unit 100. Specifically, for this purpose, the receiving member 180 may include a first insertion part 184 and a second insertion part 185.

The first insertion part 184 may be formed to pass through the bottom surface of the inlet part 181 and allow the first connection terminal 261 to be electrically connected to the electrode assembly 170.

The second insertion part 185 may be formed to pass through the bottom surface of the inlet part 181. The second insertion part 185 may be formed in an arc shape so that the second connection terminal 262 inserted therein can move from one end to the other end when the handle unit 200 and the head unit 100 rotate relative to each other. Specifically, the shape of the second insertion part 185 may be formed of a radius of curvature corresponding to the movement trajectory of the second connection terminal 262 while having a width equal or larger than the diameter of the second connection terminal 262. The second insertion part 185 allows the second connection terminal 262 to be electrically connected to the electrode assembly 170.

The second insertion part 185 may be one or more. When the number of second connection terminals 262 is two, the number of second insertion parts 185 may also be two. The two second insertion parts 185 may be formed to be symmetric with respect to the first insertion part 184. When the handle unit 200 and the head unit 100 are combined, each of the two second connection terminals 262 may pass through each of the two second insertion parts 185 and come into contact with the electrode assembly 170. Also, the first connection terminal 261 may pass through the first insertion part 184 and come into contact with the electrode assembly 170.

Hereinafter, a process in which the insertion member 210 is inserted into the receiving member 180 and thereby the handle unit 200 and the head unit 100 are combined will be described.

First, returning to FIG. 2, in a state where the handle unit 200 and the head unit 100 are positioned in a line, the user moves the handle unit 200 in the first direction and inserts the tip of the handle unit 200 into the head unit 100.

Meanwhile, referring to FIG. 10, the above-described second insertion part 185 may have a first region 185a, a second region 185b, and a position fixing protrusion 185c.

The first region 185a is a region where the one or more second connection terminals 260 are positioned when the insertion member 210 is inserted into the inlet part 181 of the receiving member 180. As described above, the protrusion portion 212 of the insertion member 210 can be inserted into only an insertion target region M while being not inserted into the other regions of the receiving member 180 because of the support part 183. Therefore, the second connection terminal 262 may be located in the first region 185a of the second insertion part 185.

The second region 185b is a region where the at least one second connection terminal 260 moved from the first region 185a is finally positioned as shown in FIG. 11 when the insertion member 210 is inserted into the inlet part 181 of the receiving member 180 and then rotated at a certain angle. Also, the second region 185b is positioned to face a middle portion of the contact part 172c formed to protrude toward the plurality of connection terminals 260.

Accordingly, in a state of being located in the second region 185b, the second connection terminal 260 may press the protruding middle portion of the contact part 172c, and thus the contact part 172c can remain in strong contact with the second connection terminal 260 while being elastically deformed.

The position fixing protrusion 185c protrudes from a portion of the second insertion part 185 where the second region 185b is located. If an external force equal to or greater than a threshold is applied in a state where the second connection terminal 262 is located in the second region 185b, the position fixing protrusion 185c enables the second connection terminal 260 to be separated from the second region 185b. If an external force equal to or greater than a threshold is not applied, the second connection terminal 262 may be continuously positioned in the second region 185b. By the position fixing protrusion 185c, a combination between the second connection terminal 262 and the receiving member 180 can be stably maintained.

Next, when the user rotates the handle unit 200 in the second direction (clockwise direction) (see FIG. 2), the head unit 100 may relatively rotate counterclockwise with respect to the handle unit 200.

When the insertion member 210 is rotated and the second connection terminal 262 is located in a second region 185b of the second insertion part 185, the rotation of the insertion member 210 may be stopped. At this time, the protrusion portion 212 may be placed under the separation preventing part 182, and the separation preventing part 182 and the protrusion portion 212 may overlap with each other. That is, even if the user pulls the handle unit 200 and the head unit 100 in the longitudinal direction, they may not be separated from each other because the protrusion portions 212 located on both sides of the insertion member 210 are respectively fitted into the corresponding separation preventing parts 182 of the receiving member 180.

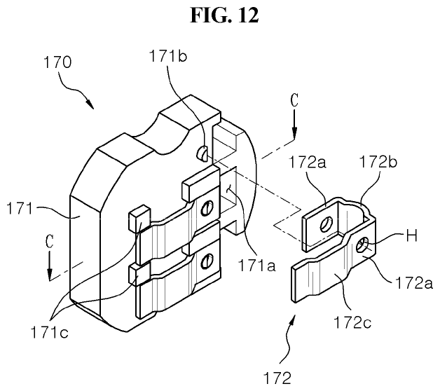

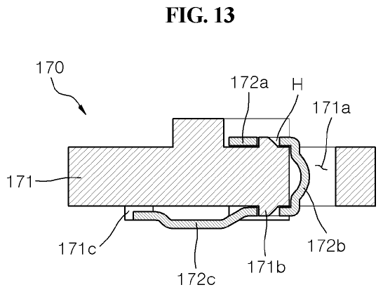

FIG. 12 is a view showing an electrode assembly extracted from the head unit of FIG. 9, and FIG. 13 is a cross-sectional view taken along line C-C in the electrode assembly of FIG. 12.

Referring to FIGS. 12 and 13, the electrode assembly 170 may include, for example, an insulating member 171 and a conductive member 172.

The insulating member 171 may be made of a non-conductive material and may have a through-hole 171a into which a portion of the auxiliary circuit board 140 is inserted.

The conductive member 172 may be made of a conductive material. One side of the conductive member 172 may be installed near the through-hole 171a of the insulating member 171 and electrically connected to the auxiliary circuit board 140 through the extension 141 inserted into the through-hole 171a. The other side of the conductive member 172 may be positioned adjacent to the receiving member 180 and electrically connected to the plurality of connection terminals 260 when the handle unit 200 and the head unit 100 are combined with each other. The conductive member 172 may be one or more.

When there are a plurality of conductive members 172, the plurality of conductive members 172 may be spaced apart from each other. The conductive member 172 may be, for example, a ribbon shape and bent several times.

The conductive member 172 may be made of an elastically deformable material such as a plate spring. Therefore, combining force with the insulating member 171 may be improved, and contact stability upon contact with the connection terminal 260 may be improved.

The number of conductive members 172 may correspond to the number of connection terminals 260. For example, when the number of connection terminals 260 is three, the number of conductive members 172 may also be three, and the connection terminals 260 and the conductive member 172 may be in contact with each other in a one-to-one correspondence.

On the other hand, the insulating member 171 may be formed with a spacer 171c and a locking jaw 171b, which can prevent adjacent conductive members 172 from coming into contact with each other. The spacer 171c may protrude from the outer surface of the insulating member 171 to be positioned between the conductive members 172 adjacent to each other. When the conductive member 172 is combined with the insulating member 171, the locking jaw 171b may be inserted into an installation hole H formed in the conductive member 172.

The conductive member 172 may continuously maintain the initial installation position by the spacer 171c and the locking jaw 171b.

The conductive member 172 may include, for example, a gripping part 172a, a pressing part 172b, and a contact part 172c.

The gripping part 172a may grip a portion of the insulating member 171 where the through-hole 171a is formed. The gripping part 172a may have, for example, but is not limited to, a shape similar to the letter ‘U’. The above-described installation hole H may be formed to pass through the gripping part 172a.

The pressing part 172b may be formed by bending a portion of the gripping part 172a that contacts the auxiliary circuit board 140, and may press the auxiliary circuit board 140.

The pressing part 172b may have an arc shape and may be formed in a portion where the through-hole 171a is located in the gripping part 172a.

In a process of assembling the head unit 100, a part (e.g., the extension 141) of the auxiliary circuit board 140 may be inserted into the through-hole 171a of the insulating member 171, and the pressing part 172b may press the auxiliary circuit board 140. Therefore, the pressing part 172b may be electrically connected to an electrode pattern (not shown) formed on the auxiliary circuit board 140. That is, the pressing part 172b not only improves the combining force between the auxiliary circuit board 140 and the electrode assembly 170, but also allows the auxiliary circuit board 140 and the conductive member 172 to be electrically connected.

The contact part 172c may extend from the gripping part 172a and come into contact with the connection terminal 260. An end portion of the contact part 172c may be spaced apart from the insulating member 171 in an unfixed state. Even if the connection terminal 260 presses the contact part 172c when the handle unit 200 and the head unit 100 are combined, a portion of the contact part 172c may be deformed to reduce the load generated on the connection terminal 260. In addition, when the handle unit 200 and the head unit 100 are combined, contact between the connection terminal 260 and the conductive member 172 can be stably maintained.

In addition, the middle portion of the contact part 172c may protrude toward the plurality of connection terminals 260. Therefore, when the handle unit 200 and the head unit 100 are combined, the contact part 172c is elastically deformed to generate an elastic force, thereby further improving the contact reliability between the connection terminal 260 and the contact part 172c.

As described above, the head unit 100 does not use wires to electrically connect the first and second electrodes 110 and 120 and the electrode assembly 170, but the auxiliary circuit board 140 may be directly fastened to the electrode assembly 170. Therefore, as a worker does not have to place wires inside the head unit 100 in a process of assembling the head unit 100, the assembly process can be more easily performed.

If components are connected using wires unlike the above, the electric toothbrush may not operate due to a short circuit occurring at a connection portion between the wires and the components or an open circuit occurring inside the electric wire. However, according to the embodiment of the present disclosure, in the head unit 100 included in the electric toothbrush 1, the auxiliary circuit board 140 and the electrode assembly 170 are mechanically fastened to each other, thereby preventing the above problem.

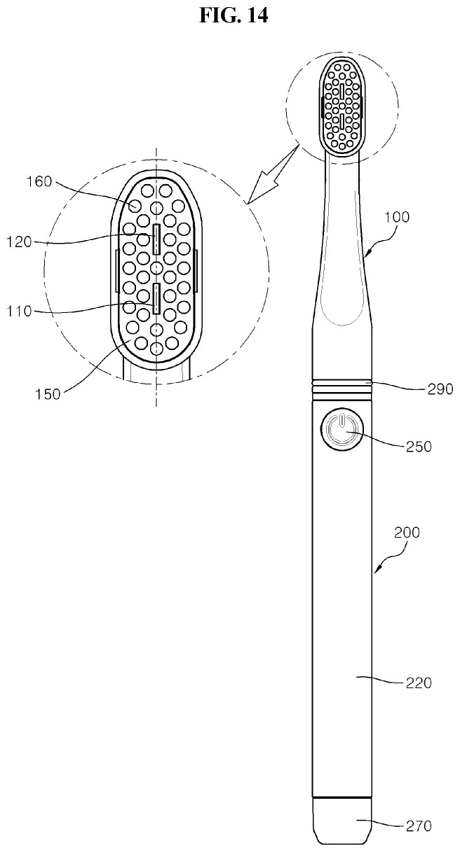

FIG. 14 is a front view showing an electric toothbrush according to an embodiment of the present disclosure.

Referring to FIG. 14, in the above-described head unit 100, the first electrode 110 and the second electrode 120 may be located to be spaced apart from each other along the length direction of the auxiliary circuit board 140. That is, based on the direction shown in the drawing, the first electrode 110 and the second electrode 120 may be positioned in a line on a virtual reference line.

The bristles 160 may be more densely disposed in a central portion of the head cover 150 than in upper and lower portions for effective removal of plaque. In addition, it is difficult to form a bristle hole (not shown) around a portion of the head cover 150 where the first electrode 110 and the second electrode 120 are positioned.

Thus, if the first and second electrodes 110 and 120 are disposed side by side in left and right directions, there is a lot of wasted space in the central portion of the head cover 150 compared to the case where the first and second electrodes 110 and 120 are arranged in a line on a virtual reference line. Therefore, it may be difficult to install the bristles 160 relatively densely because it is not possible to form many bristle holes in the head cover 150.

However, the first and second electrodes 110 and 120 included in the head unit 100 of the electric toothbrush 1 according to the embodiment of the present disclosure are positioned to be spaced apart from each other along the length direction of the auxiliary circuit board 140 and allow the bristles 160 to be installed relatively densely in the head cover 150. This can further improve the plaque removal effect.

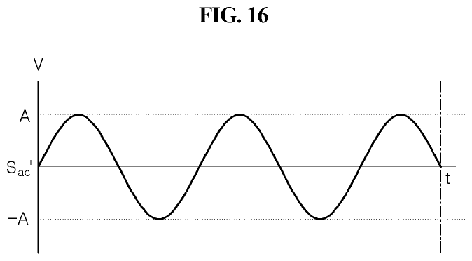

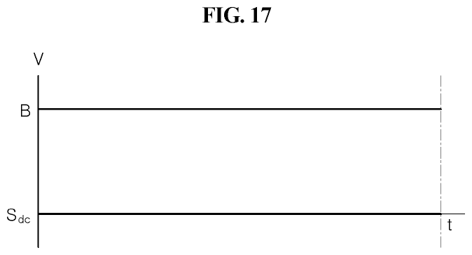

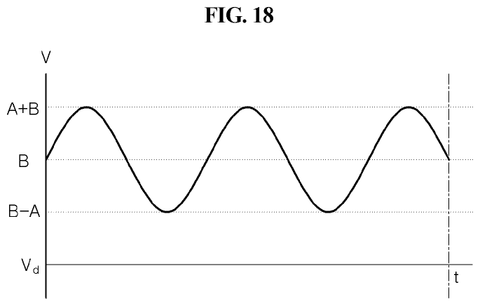

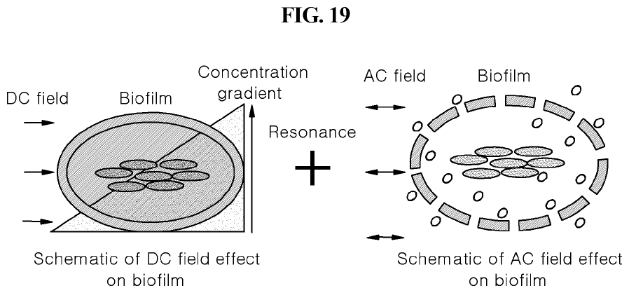

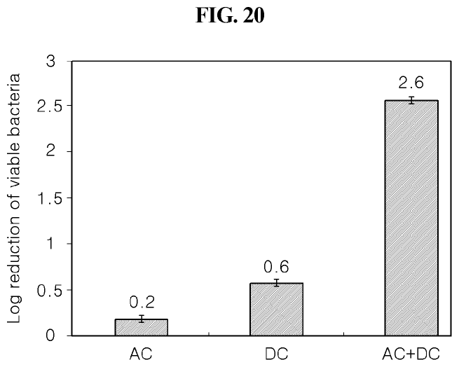

FIG. 15 is a view showing a signal supply unit mounted on a main circuit board, and FIGS. 16 to 18 are views showing waveforms of signals according to embodiments of the present disclosure. In particular, FIG. 16 shows a filtered AC signal (Sac′), FIG. 17 shows a DC signal (Sdc), and FIG. 18 shows a driving signal (Vd) generated by mixing the filtered AC signal (Sac′) and the DC signal (Sdc). In addition, FIGS. 19 and 20 are views showing a biofilm removal effect of a driving signal generated by mixing an AC signal and a DC signal.

Referring to FIG. 15, the signal supply unit SG1 is mounted on the main circuit board 240 (see FIG. 5) and can generate a driving signal (Vd) by using a battery voltage (Vb) supplied from the battery 230.

In particular, the signal supply unit SG1 can generate the driving signal (Vd) by mixing an alternating current (AC) signal and a direct current (DC) signal.

Accordingly, the driving signal (Vd) has both AC and DC components, and synergistic effects and resonance occur because the AC and DC components are simultaneously applied. Therefore, the effect of removing biofilm, which is the cause of tongue staining and bacterial film on the roof of the mouth, can be increased.

Referring to FIG. 19, the electric field caused by the DC component can induce a local imbalance in charge distribution, thereby increasing the structural stress of the biofilm, and the electric field caused by the AC component can increase the permeability of an outer shield through the generation of specific vibrations.

The synergistic effect of the AC and DC components can be seen in FIG. 20. That is, compared to the biofilm removal effect when the electric field by the AC component and the electric field by the DC component are provided alone, it can be seen that the biofilm removal effect is far superior when the electric field by the AC component and the electric field by the DC component are overlapped and provided simultaneously.

Because the electric field by the DC component and the electric field by the AC component may be simultaneously provided through the electric field generating member by the driving signal (Vd) supplied by the signal supply unit SG1 according to the embodiment of the present disclosure, it is possible to achieve the enhanced effect of the above-described biofilm removal.

In addition, as the driving signal (Vd) is set in the form of overlapping the AC voltage and the DC voltage as described above, the risk of electric shock and the pain that may be caused to the body can be reduced, compared to the case of applying only the DC voltage.

Meanwhile, the signal supply unit SG1 according to an embodiment of the present disclosure may include, for example, a DC-DC converter SG1a, a signal generator SG1b, a filter SG1c, and a calibrator SG1d, and may further include a voltage divider SG1e.

The DC-DC converter SG1a may receive a battery voltage (Vb), convert the battery voltage (Vb) into an output voltage (Vo) of a predetermined level, and output the output voltage (Vo).

The signal generator SG1b operates based on the voltage supplied from the DC-DC converter SG1a, and may generate an AC signal (Sac) having a predetermined frequency by using the output voltage (Vo) of the DC-DC converter SG1a.

The signal generator SG1b may be implemented using a known configuration capable of generating an AC signal, such as an oscillator or a function generator.

For example, the AC signal (Sac) may be set to a frequency of 1 KHz to 1000 MHz. This is because when the AC signal (Sac) is set to a low frequency of less than 1 KHz, the biofilm removal effect is reduced, and even when the AC signal (Sac) is set to a very high frequency of more than 1000 MHz, the biofilm removal effect is reduced. Meanwhile, the frequency of the AC signal (Sac) may be set to a frequency of 5 MHz to 15 MHz suitable for the biofilm removal.

In addition, the amplitude of the AC signal (Sac) may be set to 0.1 my to 3 V. If the amplitude of the AC signal (Sac) is less than 0.1 mV, it is difficult to expect a plaque removal effect, and if the amplitude of the AC signal (Sac) exceeds 3 V, there is a concern that toxic substances may be generated due to electrolysis of body fluids.

The filter SG1b may perform a filtering operation on the AC signal (Sac) generated by the signal generator SG1a. For example, the filter SG1c may include a band pass filter and convert a sawtooth wave type AC signal (Sac) into a sine wave type AC signal (Sac′). However, the type of filter SG1c is not limited to the above, and various types of filters may be employed depending on the design structure.

The calibrator SG1d may generate the driving signal (Vd) by mixing the DC signal (Sdc) with the AC signal (Sac′) supplied through the filter SG1c. For example, the calibrator SG1d may be implemented as, but is not limited to, an operating amplifier capable of summing (or overlapping) the AC signal (Sac′) and the DC signal (Sdc).

Therefore, an offset corresponding to the DC signal (Sdc) is generated in the AC signal (Sac′), and the driving signal (Vd) having both the AC and DC components can be generated.

Because the driving signal (Vd) includes all the characteristics of the AC signal (Sac), the driving signal (Vd) may be set to a frequency of 1 KHz to 1000 MHz and may also be set to a frequency of 5 MHz to 15 MHz more suitable for the biofilm removal. In addition, the amplitude of the driving signal (Vd) may be set to 0.1 my to 3 V.

Referring to FIG. 16, the calibrator SG1d may receive an AC signal (Sac′) having an amplitude of A volt (V) from the filter SG1c, and superimpose a DC signal (Sdc) of B volt (V) as shown in FIG. 17 onto the AC signal (Sac′), thereby generating a final driving signal (Vd) as shown in FIG. 18.

In this case, the voltage value of the DC signal (Sdc) may be set equal to or greater than the amplitude of the AC signal (Sac′). Therefore, the voltage value of the driving signal (Vd) may be set to zero or more.

As a result, the DC offset value of the driving signal (Vd) may be set equal to or greater than the amplitude of the driving signal (Vd).

When the DC offset value of the driving signal (Vd) is less than the amplitude of the driving signal (Vd), an interval in which the voltage of the driving signal (Vd) has a negative value occurs, and thus the loss of electrical energy is caused.

However, when the DC offset value of the driving signal (Vd) is set equal to or greater than the amplitude of the driving signal (Vd) as in the embodiment of the present disclosure, it is possible to minimize the loss of electrical energy because the voltage of the driving signal (Vd) is always zero or more.

Meanwhile, the DC signal (Sdc) may be generated by the voltage divider SG1e.

For example, the voltage divider SG1e may receive the output voltage (Vo) of the DC-DC converter SG1a and generate the DC signal (Sdc) by performing voltage division on the output voltage (Vo).

The voltage divider SG1e may be implemented as, but is not limited to, a resistor string for distributing the output voltage (Vo).

When the output voltage (Vo) of the DC-DC converter SG1a is suitable for direct use in generating the driving signal (Vd), the output voltage (Vo) may serve as the DC signal (Sdc). In this case, the voltage divider SG1e may be omitted, and the output voltage (Vo) of the DC-DC converter SG1a may be inputted to the calibrator 440.

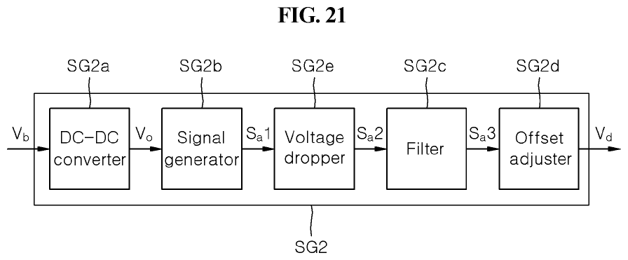

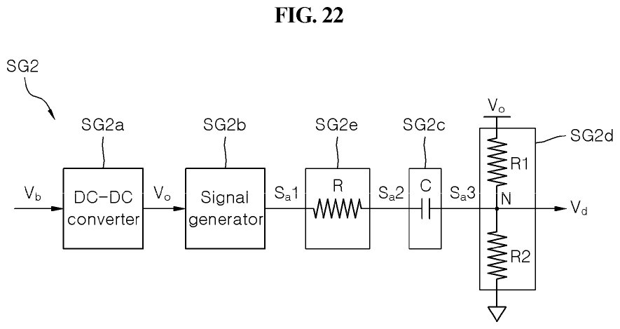

FIGS. 21 and 22 are views showing a modified example of a signal supply unit.

Referring to FIGS. 21 and 22, a signal supply unit SG2 according to a modified example of the present disclosure may include a DC-DC converter SG2a, a signal generator SG2b, a voltage dropper SG2e, a filter SG2c, and an offset adjuster SG2d.

The DC-DC converter SG2a may receive a battery voltage (Vb) from the battery 230, convert the battery voltage (Vb) into an output voltage (Vo) of a predetermined level, and output the output voltage (Vo).

The signal generator SG2b operates based on the voltage supplied from the DC-DC converter SG2a, and may generate a first AC signal (Sa1) having a predetermined frequency by using the output voltage (Vo) of the DC-DC converter SG2a.

The signal generator SG2b may be implemented using a known configuration capable of generating an AC signal, such as an oscillator or a function generator.

For example, the first AC signal (Sa1) may be set to a frequency of 1 KHz to 1000 MHz. This is because when the first AC signal (Sa1) is set to a low frequency of less than 1 KHz, the removal effect of dental plaque is reduced, and even when the first AC signal (Sa1) is set to a very high frequency of more than 1000 MHz, the removal effect of dental plaque is reduced. Meanwhile, the frequency of the first AC signal (Sa1) may be set to a frequency of 5 MHz to 15 MHz suitable for the dental plaque removal.

The voltage dropper SG2e may be used to reduce the magnitude (e.g., peak-to-peak voltage, etc.) of the first AC signal (Sa1) outputted from the signal generator SG2b. For example, the voltage dropper SG2e may be implemented as a resistor element R, and through this, a second AC signal (Sa2) whose magnitude is reduced compared to the first AC signal (Sa1) can be supplied from the voltage dropper SG2e.

The filter SG2c may perform a filtering operation on the second AC signal (Sa2) outputted from the voltage dropper SG2e. For example, the filter SG2c may be set as a low pass filter including a capacitor element C and convert the second AC signal (Sa2) of sawtooth wave type into a third AC signal (Sa3) of sine wave type.

In addition, by removing an unexpected DC offset of the second AC signal (Sa2) through the filter SG2c, it is possible to improve the accuracy of the driving signal (Vd).

The offset adjuster SG2d may generate the driving signal (Vd) by mixing the DC signal with the third AC signal (Sa3) outputted from the filter SG2c. For example, the offset adjuster SG2d may be composed of a plurality of resistor elements R1 and R2, and may generate a DC signal of a predetermined level by performing voltage division on the output voltage (Vo) of the DC-DC converter SG2a.

That is, the third AC signal (Sa3) supplied through the filter SG2c is superimposed onto the DC signal generated at a common node N of the first resistor element R1 and the second resistor element R2, so that a final driving signal (Vd) can be generated.

In this case, the amplitude of the driving signal (Vd) may be set to 0.1 my to 3 V. If the amplitude of the drive signal (Vd) is less than 0.1 mV, it is difficult to expect the plaque removal effect, and if the amplitude of the driving signal (Vd) exceeds 3 V, there is a concern that toxic substances may be generated due to electrolysis of body fluids.

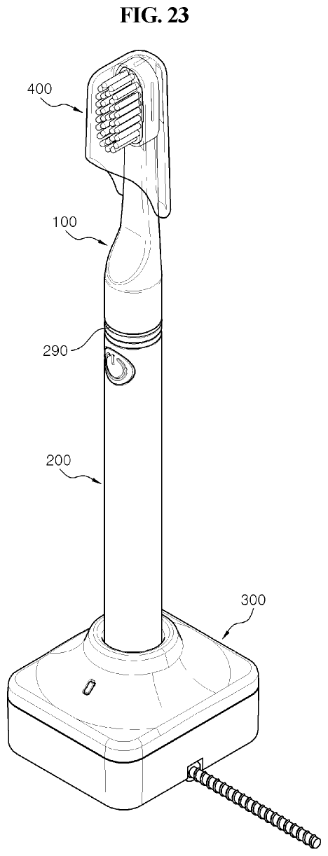

FIG. 23 is a perspective view showing a use example of an electric toothbrush according to an embodiment of the present disclosure.

Referring to FIG. 23, when the user does not use the electric toothbrush 1 according to the embodiment of the present disclosure, the head unit 100 may be covered with a protective cap 400. When the user carries the electric toothbrush 1, the protective cap 400 may protect the bristles 160 from external contaminants such as dust or foreign substances.

When charging of the electric toothbrush 1 according to the embodiment of the present disclosure is required, the user can place the electric toothbrush 1 on a charger 300. At this time, the battery 230 described above may be charged through a wireless charging scheme or a wired charging scheme.

When the electric toothbrush 1 is available for a wired charging scheme, charging terminals are provided on the outside of the handle unit 200 and on the charger 300, respectively, to come into contact with each other.

When the electric toothbrush 1 is available for a wireless charging scheme, an electromagnetic (magnetic field) induction scheme using a coil, a resonance scheme using magnetic resonance, or a radio wave radiation (RF/Micro Wave Radiation) scheme that converts electrical energy into microwaves and transmits them may be used.

The magnetic resonance scheme uses magnetic resonance (magnetic resonance phenomenon) between a transmission coil module (Tx) and a reception coil module (Rx). In this scheme, the transmission coil module (Tx) is placed in the charger 300, and the reception coil module (Rx) is disposed in the toothbrush 1, so that power is transmitted between coils.

Although certain embodiments and implementations have been described herein, other embodiments and modifications will be apparent from this description. Accordingly, the inventive concepts are not limited to such embodiments, but rather to the broader scope of the appended claims and various obvious modifications and equivalent arrangements as would be apparent to a person of ordinary skill in the art.

What is claimed is:

1. An electric toothbrush comprising:
a head unit; and
a handle unit detachably combined with the head unit and supplying a driving voltage to the head unit in response to a user's manipulation,
wherein the handle unit includes:
an insertion member including a base portion and one or more protrusion portions protruding from the base portion; and
a plurality of connection terminals having a portion positioned to protrude outward from the insertion member, and electrically connected to the head unit when the head unit and the handle unit are combined, and
wherein the head unit includes:
a receiving member including an inlet part recessed to a depth into which the insertion member is inserted, a separation preventing part protruding from an inner wall surface of the inlet part and located to overlap with the protrusion portion when the insertion member is inserted into the inlet part and then rotated at a certain angle, and a plurality of insertion parts formed to pass through a bottom surface of the inlet part; and
an electrode assembly installed adjacent to the receiving member inside the head unit, and when the handle unit and the head unit are combined, passing through the plurality of insertion parts and electrically connected to ends of the plurality of connection terminals located inside the head unit.

2. The electric toothbrush of claim 1, wherein the handle unit includes:
a handle housing combined with the insertion member;
a battery installed in the handle housing;
a main circuit board on which a signal supply unit for generating a driving voltage using a voltage of the battery is mounted; and
a connection module including a contact block made of a non-conductive material, and a contact terminal made of a conductive material and installed to surround the contact block, one side of the contact terminal being in contact with the main circuit board, and the other side being in contact with the connection terminal.

3. The electric toothbrush of claim 2, wherein the head unit includes:
a head housing including a head portion having one opened side and a combining portion communicating with the head portion;
an auxiliary circuit board installed inside the head housing;
a head cover closing an opened area of the head portion;
bristles combined with the head cover; and
first and second electrodes positioned to correspond to the head portion on the auxiliary circuit board to generate an electric field,
wherein the electrode assembly is positioned adjacent to a part of the combining portion combined with the handle unit, and is combined with the auxiliary circuit board.

4. The electric toothbrush of claim 3, wherein the electrode assembly includes:
an insulating member made of a non-conductive material and having a through-hole into which a portion of the auxiliary circuit board is inserted; and
a conductive member made of a conductive material, having one side installed near the through-hole of the insulating member and electrically connected to the auxiliary circuit board, and having the other side positioned adjacent to the receiving member and electrically connected to the plurality of connection terminals when the handle unit and the head unit are combined with each other.

5. The electric toothbrush of claim 4, wherein the conductive member includes:
a gripping part gripping a portion of the insulating member where the through-hole is formed;
a pressing part formed by bending a portion of the gripping part that contacts the auxiliary circuit board, and pressing the auxiliary circuit board; and
a contact part extending from the gripping part and coming into contact with the connection terminal.

6. The electric toothbrush of claim 5, wherein an end portion of the contact part is spaced apart from the insulating member, and
wherein a middle portion of the contact part is formed to protrude toward the plurality of connection terminals.

7. The electric toothbrush of claim 4, wherein at least one bar-shaped extension is formed in the auxiliary circuit board to be inserted into a through-hole of the insulating member.

8. The electric toothbrush of claim 3, wherein the first electrode and the second electrode are located to be spaced apart from each other along a length direction of the auxiliary circuit board.

9. The electric toothbrush of claim 3, wherein the head unit further includes a light emitting element that is installed on the auxiliary circuit board and emits light to outside.

10. The electric toothbrush of claim 3, wherein the head cover is made of a transparent material.

11. The electric toothbrush of claim 2, wherein the handle unit further includes a sealing member positioned at a portion of the handle housing that is in contact with the head unit, the sealing member sealing between the handle housing and the head unit.

12. The electric toothbrush of claim 1, wherein the plurality of connection terminals include:
a first connection terminal; and
one or more second connection terminals spaced apart from the first connection terminal.

13. The electric toothbrush of claim 12, wherein the plurality of insertion parts include:
a first insertion part formed to pass through a bottom surface of the inlet part, and allowing the first connection terminal to be electrically connected to the electrode assembly; and
at least one second insertion part formed to pass through the bottom surface of the inlet part, formed in an arc shape so that the second connection terminal inserted therein moves from one end to the other end when the handle unit and the head unit rotate relative to each other, and allowing the second connection terminal to be electrically connected to the electrode assembly.

14. The electric toothbrush of claim 13, wherein the second insertion part includes:
a first region where the one or more second connection terminals are positioned when the insertion member is inserted into the inlet part of the receiving member;
a second region positioned to face a middle portion of the contact part formed to protrude toward the plurality of connection terminals, and providing a region where the at least one second connection terminal moved from the first region is finally positioned when the insertion member is inserted into the inlet part of the receiving member and then rotated at a certain angle; and
a position fixing protrusion protruding from a portion of the second insertion part where the second region is located, wherein when an external force equal to or greater than a threshold is applied in a state where the second connection terminal is located in the second region, the position fixing protrusion enables the second connection terminal to be separated from the second region, and wherein when an external force equal to or greater than a threshold is not applied, the second connection terminal is continuously positioned in the second region.

15. A combination structure of an electric toothbrush comprising:
a head unit; and
a handle unit detachably combined with the head unit,
wherein:
the handle unit includes:
an insertion member including a plurality of protrusion portions; and
a first connection terminal and a pair of second connection terminals passing through the insertion member and exposed to outside; and
the head unit includes:
a receiving member positioned at a combining portion with the handle unit,
wherein the receiving member includes:
a plurality of separation preventing parts;
a first insertion part located in a center and into which the first connection terminal is inserted; and
a pair of second insertion parts positioned symmetrically with respect to the first insertion part and having a first region and a second region where the pair of second connection terminals are to be located,
wherein in an initial state where the insertion member is inserted into the receiving member, the second connection terminal is located in the first region, and when the handle unit rotates with respect to the head unit, the second connection terminal located in the first region moves to the second region, and the protrusion portions are respectively inserted into the separation preventing parts.
